(19) United States
(12) Patent Application Publication
(10) Pub. No.: US 2024/0293214 A1
(43) Pub. Date: Sep. 5, 2024

(54) Methods and Devices to Measure Angular Stiffness of Dental and Medical Implants

(71) Applicant: University of Washington, Seattle, WA (US)

(72) Inventors: I-Yeu Shen, Seattle, WA (US); Yifeng Liu, Seattle, WA (US); Darwin S. Wood, Seattle, WA (US); Weiwei Xu, Seattle, WA (US)

(21) Appl. No.: 18/547,897

(22) PCT Filed: Feb. 25, 2022
(86) PCT No.: PCT/US2022/017795

§ 371 Date: Aug. 25, 2023

Related U.S. Application Data
(60) Provisional application No. 63/154,968, filed on Mar. 1, 2021.

(51) Int. Cl. A61C 19/04; A61C 8/00; G16H 40/63

(52) U.S. Cl. CPC A61C 19/04; A61C 8/0089; G16H 40/63

(57) ABSTRACT

The present disclosure provides a device for measuring a stability of an implant system. The device includes a housing configured to be positioned adjacent the implant system. The device also includes an actuator coupled to the housing. The actuator is configured to vibrate the implant system when actuated. The device also includes a motion sensor coupled to the housing, and a controller in communication with the motion sensor and the actuator. The controller includes at least one processor, and data storage including program instructions stored thereon that when executed by the at least one processor, cause the controller to perform functions including: (i) receiving motion data from the motion sensor when the actuator is actuated, and (ii) determining an angular stiffness of the implant system based on the motion data.

### CROSS-REFERENCE TO RELATED APPLICATIONS

This application claims priority to U.S. Provisional Patent Application No. 63/154,968, filed Mar. 1, 2021, the contents of which are hereby incorporated by reference in their entirety.

### BACKGROUND

Unless otherwise indicated herein, the materials described in this section are not prior art to the claims in this application and are not admitted to be prior art by inclusion in this section.

When a dental implant is successfully placed in bone, the bone will naturally heal and grow around the implant providing structural and functional connection. That bond between the bone and the implant is known as osseointegration. A higher degree of osseointegration implies a stronger bond and immobility of the implant and thus higher stability. Therefore, the degree of osseointegration is loosely called “stability” in dentistry. Due to various reasons (e.g., low bone density or complications from surgeries), an implant may not have enough stability to receive a bite load or even to be functional. Therefore, measurements and quantification of implant stability have always been a major research issue in periodontics.

To quantify dental implant stability, there are two specific challenges to overcome. The first challenge is to identify a physical parameter that can effectively and accurately represent the stability. The second challenge is to accurately measure the physical parameter that quantifies implant stability in a clinical environment. Due to these issues and challenges, there are very few commercially available products to measure implant stability.

### SUMMARY

The present disclosure provides a device to measure the stability of a dental implant in a clinical environment.

Accordingly, in one embodiment the present disclosure provides a device for measuring a stability of an implant system, the device comprising: a housing configured to be positioned adjacent the implant system; an actuator coupled to the housing, wherein the actuator is configured to vibrate the implant system when actuated; a motion sensor coupled to the housing; and a controller in communication with the motion sensor and the actuator, wherein the controller includes at least one processor, and data storage including program instructions stored thereon that when executed by the at least one processor, cause the controller to perform functions including: (i) receiving motion data from the motion sensor when the actuator is actuated, and (ii) determining an angular stiffness of the implant system based on the motion data.

In another embodiment, the motion sensor comprises an accelerometer, and wherein the motion data comprises acceleration data.

In another embodiment, the implant system includes an implant implanted in a bone and an abutment coupled to the implant, wherein at least a portion of the abutment is exposed and not directly coupled to the bone.

In another embodiment, the housing includes a cutout, and wherein the abutment is configured to be positioned at least partially within the cutout.

In another embodiment, the implant system includes a longitudinal axis extending from a first surface to a second surface opposite the first surface, wherein the implant system includes a second axis that is perpendicular to the longitudinal axis, and wherein the angular stiffness corresponds to a stiffness of a rotation of the implant system with respect to the second axis.

In another embodiment, the controller is further configured to: provide a binary indication of whether or not the implant system is stable based on the determined angular stiffness of the implant system.

In another embodiment, the controller is further configured to: provide a notification of a degree of stability of the implant system based on the determined angular stiffness of the implant system.

In another embodiment, the actuator is configured to vibrate at a frequency below a resonance frequency of the implant system.

In another embodiment, the actuator is configured to vibrate at a first frequency which is measured by the motion sensor to define a first motion data, wherein the actuator is configured to vibrate at a second frequency which is measured by the motion sensor to define a second motion data, and wherein the user interface determines the angular stiffness of the implant system based on both the first motion data and the second motion data.

In another embodiment, the actuator comprises a first actuator, the device further comprising: a second actuator coupled to the housing, wherein the second actuator is configured to vibrate the implant system when actuated.

In another embodiment, the first actuator is configured to vibrate at a first frequency which is measured by the motion sensor to define a first motion data, wherein the second actuator is configured to vibrate at a second frequency which is measured by the motion sensor to define a second motion data, and wherein the user interface determines the angular stiffness of the implant system based on both the first motion data and the second motion data.

In another embodiment, the actuator comprises a buzzer motor.

In another embodiment, the actuator comprises a piezoelectric actuator.

In another embodiment, the actuator is driven sinusoidally such that the motion data comprises digitized sinusoidal signals.

In another embodiment, the implant system comprises one of a dental implant, an abutment, a dental crown, a dental restoration, a bone screw, a plate, a hip implant, or a knee implant.

In another embodiment, the device further includes one or more bypass capacitors positioned between the motion sensor and the controller.

In another embodiment, determining an angular stiffness of the implant system based on the motion data comprises: transmitting the motion data to a user interface, wherein the user interface is configured to determine the angular stiffness of the implant system based on the motion data.

In another embodiment, the motion data comprises acceleration data, and wherein determining the angular stiffness of the implant system based on the acceleration data comprises: applying a nonlinear regression algorithm to extract a frequency ω and an amplitude A₀ of the acceleration data; determining an experimentally measured flexibility (C_{AB})ₑₓₚ of the implant system using the equation

$${( {C}_{A B} )}_{exp} = \frac{1}{{ω}^{2}} ( {A}_{0} / {F}_{0} ) ,$$

wherein F₀ is an amplitude of an actuator force; and determining the angular stiffness (k_{θ}) using the interpolation

$${k}_{θ} = {( {k}_{θ} )}_{i} + \frac{{( {C}_{A B} )}_{e x p} - {( {C}_{A B} )}_{i}}{{( {C}_{A B} )}_{i + 1} - {( {C}_{A B} )}_{i}} [ {( {k}_{θ} )}_{i + 1} - {( {k}_{θ} )}_{i} ] ,$$

wherein (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

In another embodiment, the present disclosure provides a device for measuring a stability of an implant system, the device comprising: a housing configured to be positioned adjacent the implant system; an actuator coupled to the housing, wherein the actuator is configured to vibrate the implant system when actuated; a motion sensor coupled to the housing; and a controller in communication with the motion sensor and the actuator, wherein the controller includes at least one processor, and data storage including program instructions stored thereon that when executed by the at least one processor, cause the controller to perform functions including: (i) receiving motion data from the motion sensor when the actuator is actuated, and (ii) transmitting the motion data to a user interface, wherein the user interface is configured to determine an angular stiffness of the implant system based on the motion data.

In another embodiment, the motion sensor comprises an accelerometer, and wherein the motion data comprises acceleration data.

In another embodiment, the implant system includes an implant implanted in a bone and an abutment coupled to the implant, wherein at least a portion of the abutment is exposed and not directly coupled to the bone.

In another embodiment, the housing includes a cutout, and wherein the abutment is configured to be positioned at least partially within the cutout.

In another embodiment, the implant system includes a longitudinal axis extending from a first surface to a second surface opposite the first surface, wherein the implant system includes a second axis that is perpendicular to the longitudinal axis, and wherein the angular stiffness corresponds to a stiffness of a rotation of the implant system with respect to the second axis.

In another embodiment, the user interface is further configured to: provide a binary indication of whether or not the implant system is stable based on the determined angular stiffness of the implant system.

In another embodiment, the user interface is further configured to: provide a notification of a degree of stability of the implant system based on the determined angular stiffness of the implant system.

In another embodiment, the actuator is configured to vibrate at a frequency below a resonance frequency of the implant system.

In another embodiment, the actuator is configured to vibrate at a first frequency which is measured by the motion sensor to define a first motion data, wherein the actuator is configured to vibrate at a second frequency which is measured by the motion sensor to define a second motion data, and wherein the user interface determines the angular stiffness of the implant system based on both the first motion data and the second motion data.

In another embodiment, the actuator comprises a first actuator, the device further comprising: a second actuator coupled to the housing, wherein the second actuator is configured to vibrate the implant system when actuated.

In another embodiment, the first actuator is configured to vibrate at a first frequency which is measured by the motion sensor to define a first motion data, wherein the second actuator is configured to vibrate at a second frequency which is measured by the motion sensor to define a second motion data, and wherein the user interface determines the angular stiffness of the implant system based on both the first motion data and the second motion data.

In another embodiment, the actuator comprises a buzzer motor.

In another embodiment, the actuator comprises a piezoelectric actuator.

In another embodiment, the actuator is driven sinusoidally such that the motion data comprises digitized sinusoidal signals.

In another embodiment, the implant system comprises one of a dental implant, an abutment, a dental crown, a dental restoration, a bone screw, a plate, a hip implant, or a knee implant.

In another embodiment, the device further includes one or more bypass capacitors positioned between the motion sensor and the controller.

In another embodiment, the motion data comprises acceleration data, and wherein determining the angular stiffness of the implant system based on the acceleration data comprises: applying a nonlinear regression algorithm to extract a frequency ω and an amplitude A₀ of the acceleration data; determining an experimentally measured flexibility (C_{AB})ₑₓₚ of the implant system using the equation

$${( {C}_{A B} )}_{exp} = \frac{1}{{ω}^{2}} ( {A}_{0} / {F}_{0} ) ,$$

wherein F₀ is an amplitude of an actuator force; and determining the angular stiffness (k_{θ}) using the interpolation

$${k}_{θ} = {( {k}_{θ} )}_{i} + \frac{{( {C}_{A B} )}_{e x p} - {( {C}_{A B} )}_{i}}{{( {C}_{A B} )}_{i + 1} - {( {C}_{A B} )}_{i}} [ {( {k}_{θ} )}_{i + 1} - {( {k}_{θ} )}_{i} ] ,$$

wherein (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

In another embodiment, the present disclosure provides a method for measuring a stability of an implant system, the method comprising: positioning a device adjacent the implant system, wherein the device comprises (i) a housing, (ii) an actuator coupled to the housing, and (iii) a motion sensor coupled to the housing; actuating the actuator of the device to vibrate the implant system; determining, via the motion sensor, motion data of the implant system; and determining an angular stiffness of the implant system based on the motion data.

In another embodiment, determining the angular stiffness of the implant system comprises: receiving, via a controller of the device, the motion data of the implant system; transmitting, via the controller, the motion data to a user interface; and determining, via the user interface, the angular stiffness of the implant system based on the motion data.

In another embodiment, the motion data comprises acceleration data, and wherein determining the angular stiffness of the implant system based on the acceleration data comprises: applying a nonlinear regression algorithm to extract a frequency ω and an amplitude A₀ of the acceleration data; determining an experimentally measured flexibility (C_{AB})ₑₓₚ of the implant system using the equation

$${( {C}_{A B} )}_{exp} = \frac{1}{{ω}^{2}} ( {A}_{0} / {F}_{0} ) ,$$

wherein F₀ is an amplitude of an actuator force; and determining the angular stiffness (k_{θ}) using the interpolation

$${k}_{θ} = {( {k}_{θ} )}_{i} + \frac{{( {C}_{A B} )}_{e x p} - {( {C}_{A B} )}_{i}}{{( {C}_{A B} )}_{i + 1} - {( {C}_{A B} )}_{i}} [ {( {k}_{θ} )}_{i + 1} - {( {k}_{θ} )}_{i} ] ,$$

where (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a dental implant to illustrate the operating principle, according to an example embodiment.

FIG. 2A illustrates a finite element model of a dental implant, according to an example embodiment.

FIG. 2B illustrates a cross-sectional view of the finite element model of FIG. 2A, according to an example embodiment.

FIG. 2C illustrates a cross-sectional view of the implant and abutment of the finite element model of FIG. 2A, according to an example embodiment.

FIG. 2D illustrates a graph showing an interpolation to determine an angular stiffness of the finite element model of FIG. 2A, according to an example embodiment.

FIG. 3 illustrates a device for measuring a stability of an implant system, according to an example embodiment.

FIG. 4 illustrates a flowchart of the user interface of the device, according to an example embodiment.

FIG. 5 illustrates a graph showing extraction of frequency ω and amplitude A₀ using nonlinear regression, according to an example embodiment.

FIG. 6 illustrates a graph showing motor speed ω, displacement X₀, and angular stiffness k_{θ} during a 5-minute test, according to an example embodiment.

FIG. 7 illustrates a graph showing extracted angular stiffness k_{θ} for four motor runs, according to an example embodiment.

FIGS. 8A-8D illustrate a process flow for a benchmarking process, according to an example embodiment.

FIG. 9 is a flowchart illustrating an example method for measuring a stability of an implant system, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

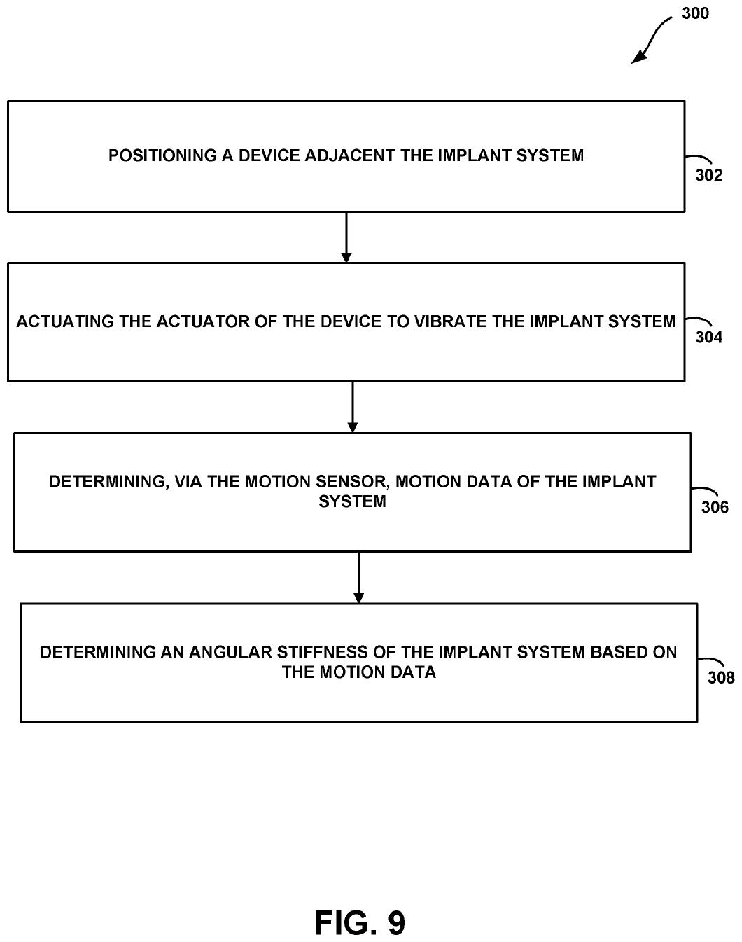

In FIG. 9, referred to above, the blocks may represent operations and/or portions thereof and lines connecting the various blocks do not imply any particular order or dependency of the operations or portions thereof. It will be understood that not all dependencies among the various disclosed operations are necessarily represented. FIG. 9 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means +/−5%.

As used herein, with respect to measurements, “substantially” means +/−5%.

Generally, the present disclosure provides a device and methods of use thereof to measure the stability of a dental implant in a clinical environment.

Stability of a dental implant reflects quality of osseointegration between the implant and its surrounding bone. While many methods have been proposed to characterize implant stability, angular stiffness at the neck of the implant has been proven to be a rigorous and accurate measure. Nevertheless, fast and reliable measurements of the angular stiffness in a clinical environment is not yet available. The present disclosure provides a novel stability diagnostic device to measure the angular stiffness accurately in clinical environments. The device consists of a motion sensor, a controller, and a user interface. In the sensing unit, a housing attaches an actuator and a motion sensor to an abutment of an implant, whose angular stiffness is to be measured. The actuator vibrates at a frequency below the resonance frequency of the implant-bone-abutment system. Meanwhile, the motion sensor measures motion data of the abutment. The controller controls the actuator, reads the motion data, and transmits the motion data to the user interface via a wired or wireless link. The user interface may post-process the data and extract the angular stiffness through use of a finite element model and a nonlinear regression algorithm. The extracted angular stiffness may be benchmarked against that obtained via a force hammer and a laser Doppler vibrometer.

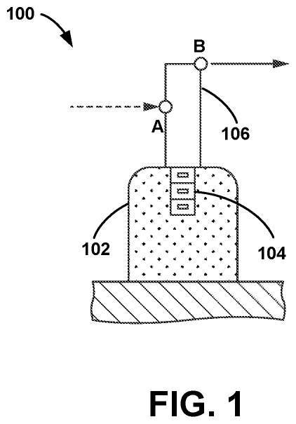

FIG. 1 shows a dental implant model 100 to illustrate the operating principles. The model 100 includes an elastic support 102, an implant 104, and an abutment 106. The elastic support 102 is assumed to be isotropic with Young's modulus E and Poisson ratio v. In some cases, the elastic support 102 is to model a foundation that receives the implant 104. In a clinical environment, the elastic support 102 could be the bone structure in mandible or maxilla. In a lab environment, the elastic support 102 could be artificial bones or cow bones clamped in a vice. The implant 104 is placed in the elastic support 102 at a known location. The implant 104 is assumed to be perfectly bonded to the elastic support 102. Moreover, the implant 104 has known geometry, dimensions, and material properties (e.g., titanium). Finally, the abutment 106 is the portion above the implant 104. It could be a healing abutment, impression coping, a locator abutment, or a crown. Usually, its material properties and geometry are also known.

The model 100 described above is a good compromise between practicality and complex clinical environments. On the one hand, this model is simple enough so that the angular stiffness can be found easily and reliably. On the other hand, the model 100 is versatile enough to accommodate complex scenarios encountered in clinical environments. For example, bones are anisotropic and their material properties vary widely among people. The bonding between the bone and the implant may not be perfect (e.g., partial bonding). All these scenarios can be incorporated by interpreting E as an equivalent Young's modulus of the model 100. This is, in essence, the concept of homogenization in micromechanics and in composite materials.

With the model 100 in FIG. 1, the goal is to extract the angular stiffness k_{θ} at the implant-abutment junction while the equivalent Young's modulus E of the elastic support is experimentally unknown. To attain this goal, a three-step approach is devised as follows.

The first step is to obtain flexibility of the support-implant-abutment system experimentally. To do so, a harmonic force

$$F ( t ) = {F}_{0} cos ω t \qquad ( 1 )$$

is applied to point B on the abutment (as shown in FIG. 1), where F₀ is the amplitude and ω is the driving frequency of the force. Specifically, the driving frequency ω is significantly less than the first natural frequency (or resonance frequency) ω₁ of the support-implant-abutment system, i.e., ω«ω₁. As a result, the system is quasi-static and inertial forces can be ignored. Also, the displacement x(t) of a point A on the abutment will be in phase with F(t) because ω«ω₁, i.e.,

$$x ( t ) = {X}_{0} cos ω t \qquad ( 2 )$$

where X₀ is the amplitude. Moreover, F₀ and X₀ are governed by linear elasticity because the system is quasi-static and the applied force is small.

In experiments, it is easier to measure acceleration instead. According to (2), acceleration a(t) at point A is

$$a ( t ) = \ddot{x} ( t ) ≡ - {A}_{0} cos ω t \qquad ( 3 )$$

where the acceleration amplitude A₀ is given as

$${A}_{0} = {X}_{0} {ω}^{2} \qquad ( 4 )$$

Experimentally, if a known force F(t) in (1) is applied and the acceleration a(t) in (3) is measured, one can extract the flexibility or compliance (C_{AB})ₑₓₚ from points B to A as

$${( {C}_{AB} )}_{exp} ≡ {X}_{0} / {F}_{0} = \frac{1}{{ω}^{2}} ( {A}_{0} / {F}_{0} ) \qquad ( 5 )$$

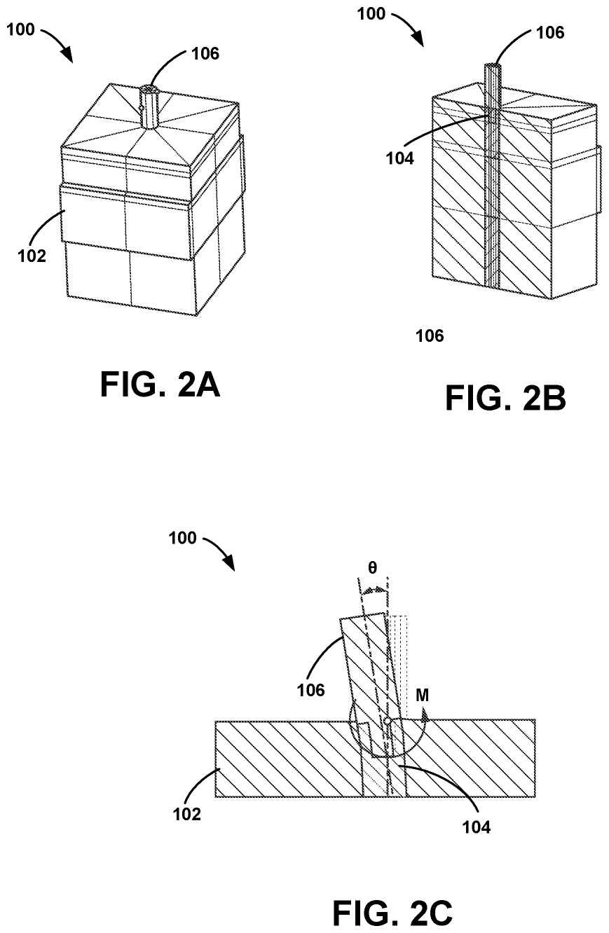

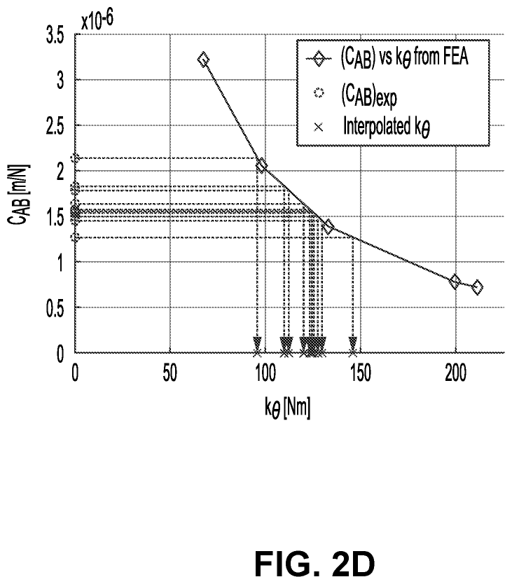

The second step is to determine the equivalent Young's modulus E based on the experimentally measured flexibility (C_{AB})ₑₓₚ from (5). This can be done through use of finite element simulations and interpolation as follows. A finite element model is first created to mimic geometry and dimensions of the actual elastic support, implant, and abutment used in the test. For example, if an implant is placed in mandible and the flexibility (C_{AB})ₑₓₚ is measured via an impression coping in a clinical environment, the finite element model will include the implant, the impression coping (as the abutment), and a homogeneous mandible whose equivalent Young's modulus E is to be determined. As another example, FIG. 2A shows a finite element model to mimic a lab experiment. The model includes a titanium alloys cylindrical abutment, a titanium implant, and a block of homogenous material clamped at two opposite sides. The equivalent Young's modulus E of the block, however, is to be determined from the measured flexibility (C_{AB})ₑₓₚ.

One can use a trial-and-error process to extract the equivalent Young's modulus E as follows. When the block is trialed with a first Young's modulus E₁, a unit force is applied to point B. The corresponding static deflection at point A (e.g., from a static finite element analysis) gives a first theoretical flexibility (C_{AB})₁. Next, the block is trialed with a second (and increased) Young's modulus E₂ to obtain a second theoretical flexibility (C_{AB})₂. The trial-and-error process proceeds with ascending trialed Young's moduli Eᵢ, i=1, 2, 3, . . . , to obtain the corresponding theoretical flexibilities (C_{AB})ᵢ, which are descending in numerical values. The trial-and-error process ends when the measured flexibility (C_{AB})ₑₓₚ falls between two consecutive theoretical flexibilities (C_{AB})ᵢ and (C_{AB})ᵢ₊₁, i.e.,

$${( {C}_{AB} )}_{i} > {( {C}_{AB} )}_{exp} > {( {C}_{AB} )}_{i + 1} \qquad ( 6 )$$

The equivalent Young's modulus E is then obtained via linear interpolation as

$$E = {E}_{i} + \frac{{( {C}_{AB} )}_{exp} - {( {C}_{AB} )}_{i}}{{( {C}_{AB} )}_{i + 1} - {( {C}_{AB} )}_{i}} ( {E}_{i + 1} - {E}_{i} ) \qquad ( 7 )$$

The third step is to extract the angular stiffness k_{θ} corresponding to the equivalent Young's modulus E. This can also be done through finite element modeling. For the example of FIG. 2A, the equivalent Young's modulus E from (7) is first used in the finite element model for the block. Next, a unit moment is applied at the implant-abutment junction, as shown in FIG. 2C. Then the angle of rotation at the tip of the abutment is 1/k_{θ}. Alternatively, one can integrate this step with the trial-and-error process above. When the trial-and-error process proceeds with ascending trialed Young's moduli Eᵢ, i=1, 2, 3, . . . , one can obtain not only the corresponding flexibility (C_{AB})ᵢ but also the corresponding angular stiffness (k_{θ})ᵢ by applying a unit moment (as shown in FIG. 2C). Thus, one can obtain a relationship between (C_{AB})ᵢ and (k_{θ})ᵢ as shown in FIG. 2D. Accordingly, the interpolation

$${k}_{θ} = {( {k}_{θ} )}_{i} + \frac{{( {C}_{AB} )}_{exp} - {( {C}_{AB} )}_{i}}{{( {C}_{AB} )}_{i + 1} - {( {C}_{AB} )}_{i}} [ {( {k}_{θ} )}_{i + 1} - {( {k}_{θ} )}_{i} ] \qquad ( 8 )$$

gives the angular stiffness k_{θ} corresponding to the measured flexibility (C_{AB})ₑₓₚ.

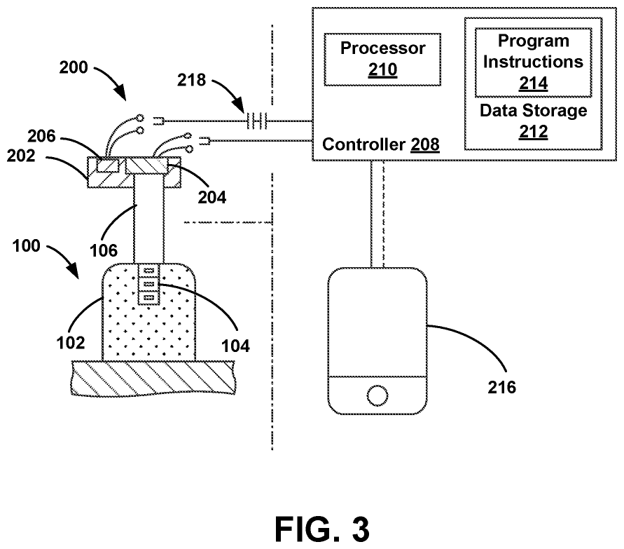

Based on the operating principles described above and with reference to FIG. 3, a device 200 for measuring a stability of an implant system 100 is shown and described. As shown in FIG. 3, the device 200 includes a housing 202 configured to be positioned adjacent the implant system 100. The device 200 further includes an actuator 204 coupled to the housing 202. The actuator 204 is configured to vibrate the implant system 100 when actuated. The actuator is configured to vibrate at a frequency below a resonance frequency of the implant system 100. The device 200 further includes a motion sensor 206 coupled to the housing 202. The device 200 further includes a controller 208 in communication with the motion sensor 206 and the actuator 204. The controller includes at least one processor 210, and data storage 212 including program instructions 214 stored thereon that when executed by the at least one processor 210, cause the controller 208 to perform functions including: (i) receiving motion data from the motion sensor 206 when the actuator 204 is actuated, and (ii) determining an angular stiffness of the implant system 100 based on the motion data.

In one example, the step of determining an angular stiffness of the implant system 100 based on the motion data comprises transmitting the motion data to a user interface 216, and the user interface 216 is configured to determine the angular stiffness of the implant system based on the motion data. As such, the step of determining the angular stiffness of the implant system 100 based on the motion data may be performed by the controller 208 (e.g., firmware loaded in read only memory (ROM) and stored in the controller 208), or by the user interface 216 (e.g., software loaded in the user interface 216).

The controller 208 may take a variety of forms. The controller 208 can be any type of control unit including, but not limited to, a microprocessor, a microcontroller, a digital signal processor, or any combination thereof. The controller 208 can communicate with the end-user either via its USB port or its built-in Bluetooth antenna. Additionally, the controller 208 has the hardware capabilities to communicate with the motion sensor 206 (e.g., via Serial Peripheral Interface (SPI) as a non-limiting example). The main functions of the controller 208 are (a) to provide a DC voltage to drive the actuator 204, (b) to power the motion sensor 206 and to read the measured motion data, and (c) to transmit the motion data to the user interface 216 via a wired (e.g., USB) or a wireless link.

In one example, as shown in FIG. 3, the device 200 further includes a pair of bypass capacitors 218 on the output data lines of the motion sensor 206. These pair of bypass capacitors 218 may help maintain signal integrity of the motion data as it is transmitted to the controller 208.

In one example, the motion sensor 206 comprises an accelerometer (such as a MEMS accelerometer as a non-limiting example), and the motion data comprises acceleration data. In another example, the motion sensor 206 may comprise any sensor capable of measuring motion and/or vibration.

In one example, the implant system includes an implant 104 implanted in a support 102 (e.g., a bone) and an abutment 106 coupled to the implant 104. In one such example, at least a portion of the abutment 106 is exposed and not directly coupled to the bone 102. In one such example, the housing 202 includes a cutout, and the abutment 106 is configured to be positioned at least partially within the cutout. As such, in one example the housing 202 does not directly contact the implant 104 of the implant system 100. In one example, the abutment 106 is configured to be press fit into the cutout of the housing 202. In another example, a latch mechanism or a cap may be used to couple the abutment 106 to the housing 202. The implant system 100 may comprise one of a dental implant, an abutment, a dental crown, a dental restoration, a bone screw, a plate, a hip implant, or a knee implant.

In one example, the implant system 100 includes a longitudinal axis extending from a first surface to a second surface opposite the first surface, the implant system 100 includes a second axis that is perpendicular to the longitudinal axis, and the angular stiffness corresponds to a stiffness of a rotation of the implant system 100 with respect to the second axis.

In one example, the controller 208 and/or the user interface 216 is further configured to provide a binary indication of whether or not the implant system 100 is stable based on the determined angular stiffness of the implant system 100. In another example, the controller 208 and/or the user interface 216 is further configured to provide a notification of a degree of stability of the implant system 100 based on the determined angular stiffness of the implant system 100. In one particular example, the notification of a degree of stability of the implant system 100 comprises three categories: stable, unstable, and marginal. Other degrees of stability are possible as well.

In one example, the actuator 204 is configured to vibrate at a first frequency which is measured by the motion sensor 206 to define a first motion data, the actuator 204 is configured to vibrate at a second frequency which is measured by the motion sensor 206 to define a second motion data, and the controller 208 and/or the user interface 216 determines the angular stiffness of the implant system 100 based on both the first motion data and the second motion data.

In one example, the actuator 204 comprises a first actuator, and the device further comprises a second actuator coupled to the housing 202, where the second actuator is configured to vibrate the implant system 100 when actuated. In one such example, the first actuator is configured to vibrate at a first frequency which is measured by the motion sensor 206 to define a first motion data, the second actuator is configured to vibrate at a second frequency which is measured by the motion sensor 206 to define a second motion data, and the controller 208 and/or the user interface 216 determines the angular stiffness of the implant system 100 based on both the first motion data and the second motion data.

The actuator 204 may take a variety of forms. In one example, the actuator 204 comprises a buzzer motor. In one particular example, the buzzer motor has a diameter of ø8 mm and thickness of 2 mm. The buzzer motor speed is rated at 15,000±20% rpm for 3V DC. At a given speed ω, the harmonic force F₀ is found via the manufacturer's specifications and calibration. In another example, the actuator 204 comprises a piezoelectric actuator. Other actuators are possible as well. In one example, the actuator 204 is driven sinusoidally such that the motion data comprises digitized sinusoidal signals.

In one example, as discussed above, the motion data comprises acceleration data. In one such example, determining the angular stiffness of the implant system 100 based on the acceleration data comprises: (i) applying a nonlinear regression algorithm to extract a frequency ω and an amplitude A₀ of the motion data, (ii) determining an experimentally measured flexibility (C_{AB})ₑₓₚ of the implant system using the equation

$${( {C}_{AB} )}_{exp} = \frac{1}{{ω}^{2}} ( {A}_{0} / {F}_{0} ) ,$$

wherein F₀ is an amplitude of an actuator force, and (iii) determining the angular stiffness (k_{θ}) using the interpolation

$${k}_{θ} = {( {k}_{θ} )}_{i} + \frac{{( {C}_{AB} )}_{exp} - {( {C}_{AB} )}_{i}}{{( {C}_{AB} )}_{i + 1} - {( {C}_{AB} )}_{i}} [ {( {k}_{θ} )}_{i + 1} - {( {k}_{θ} )}_{i} ] ,$$

wherein (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

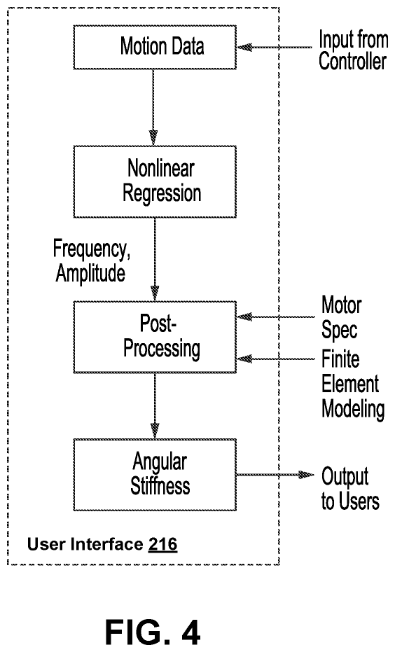

FIG. 4 illustrates the flowchart of the user interface 216. First, the programmed controller 208 reads the measured motion data from the motion sensor 206 and transmits it to the user interface 216. The next step is to process the motion data. Since the motion data comprises digitized sinusoidal signals, a nonlinear regression algorithm is used to extract the frequency ω and amplitude A₀ of the measured motion (e.g., acceleration as a non-limiting example). The last step is post-processing to extract the angular stiffness k_{θ}. The post-processing unit in the user interface 216 may be preinstalled with several tables, including (a) the harmonic force F₀ vs. the spin speed ω, and (b) the equivalent Young's modulus Eᵢ, the corresponding flexibility (C_{AB})ᵢ and angular stiffness (k_{θ})ᵢ. After the nonlinear regression algorithm extracts frequency ω and amplitude A₀, the post-processing unit calculates the experimentally measured flexibility (C_{AB})ₑₓₚ using equation (5). The post-processing unit then further extracts the angular stiffness k_{θ} using equation (8), also see FIG. 2D.

The user interface 216 may take a variety of forms. In one example, the controller 208 is integrated with and an integral part of the user interface 216. In another example, the controller 208 is separate from, but in wired or wireless communication with, the user interface 216. The user interface 216 may comprise an external device, such as a mobile phone, a tablet, a laptop, or other personal computer as examples.

The device 200 described above is unique in multiple ways. First, the housing 202, actuator 204, and motion sensor 206 make up a sensing unit that is small and may be disposable. As such, and as shown in FIG. 3, the controller 208 may be removably coupled to the actuator 204 and/or the motion sensor 206. Such an arrangement enables the housing 202, actuator 204, and motion sensor 206 to be disposable, while the controller 208 and user interface 216 can be reused. In one example, a sizing of the housing 202 is configured to allow at least a portion of the housing 202 to fit in the tiny space of a missing tooth. Second, the device 200 bypasses resonance frequency analysis (RFA) and measures implant stiffness directly. This feature is, indeed, very subtle. In fact, one way to obtain angular stiffness is to measure resonance frequencies first and extract the angular stiffness thereof via finite element models. Since the resonance frequency varies significantly with respect to boundary conditions and implant location in a mandible model, extracting angular stiffness accurately from resonance frequency measurements will not be viable in a clinical environment. To avoid this pitfall, the device 200 operates at a frequency substantially lower than the resonance frequency. In the sub-resonant frequency range, stiffness of the implant-bone interface dominates the vibratory response; therefore, the angular stiffness can be extracted accurately thereof. Third, the device 200 is able to extract angular stiffness of implant systems with high accuracy. Accurate extraction of angular stiffness from the measured acceleration data is enabled via knowledge-based processes, such as nonlinear regression and finite element modeling.

To test the device 200 described above, three buzzer motors N1, N2, and N3 were used to measure the angular stiffness k_{θ} of the implant test model. Motor N1 was tested twice on Day 1 and Day 2. Motors N2 and N3 were tested once on Day 2. Each test lasted for 5 minutes. Accelerometer data were collected continuously but analyzed every 20 seconds to extract the angular stiffness k_{θ}.

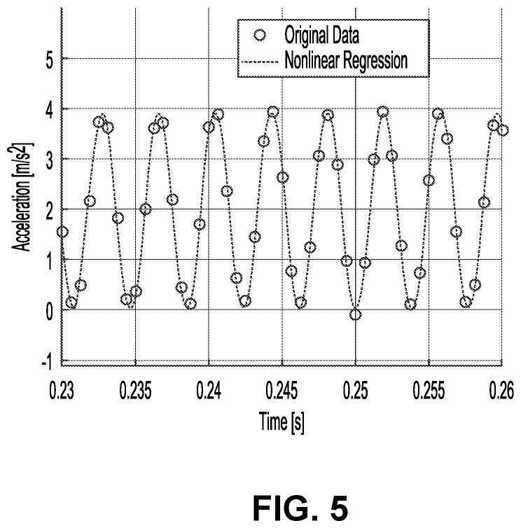

The nonlinear regression is then applied to the digitized acceleration data from the accelerometer to extract buzzer motor speed w and acceleration amplitude A₀, as shown in FIG. 5.

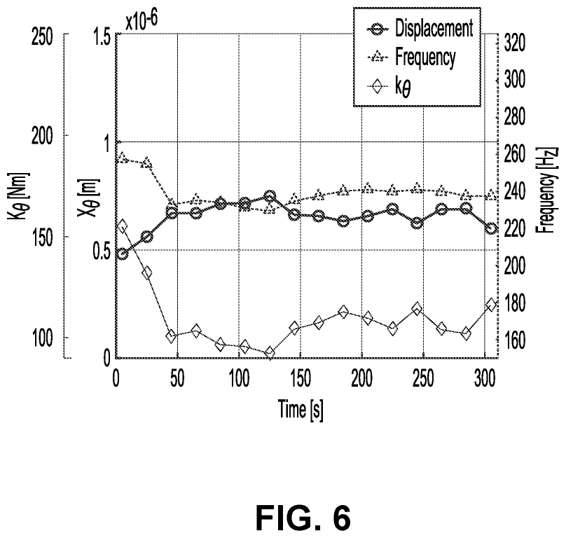

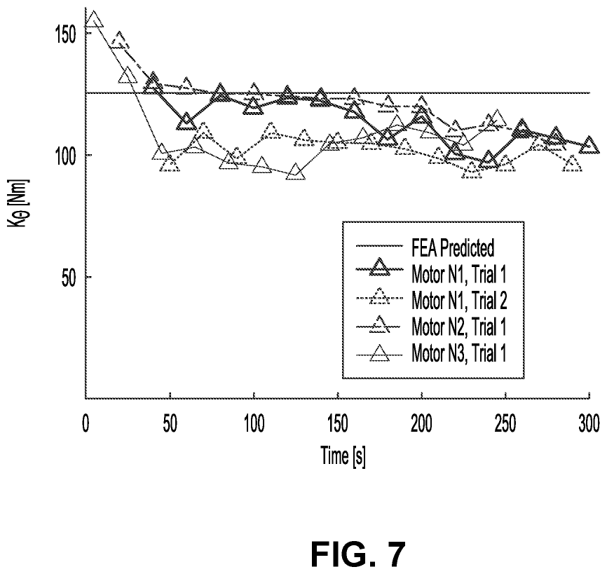

FIG. 6 shows the buzzer motor speed ω, displacement X₀, and angular stiffness k_{θ} during a 5-minute test for buzzer motor N2. The motor speed is not stable and varies between 250 Hz and 280 Hz. Between 50 and 150 seconds, the motor speed ω, displacement X₀, and angular stiffness k_{θ} are relatively stable. The average k_{θ} over the 5-minute test is 120.80 N·m. FIG. 7 shows the angular stiffness k_{θ} of four motor runs. The bulk of the extracted k_{θ} is between 100 N·m and 120 N·m, which amounts to roughly 20% variations. Moreover, Table 1 below tabulates average k_{θ} of the four motor runs for comparisons, which shows the same variations.

Although four different motor runs lead to similar angular stiffness k_{θ}, they should be benchmarked or calibrated against angular stiffness k_{θ} obtained from other measurement methods in order to validate the function and accuracy of the stability tester. Table 1 below shows the average k_{θ} of the four motor runs.

TABLE 1
Average k_{θ} of four motor runs

| | Test | Average | Benchmark | |
|---|---|---|---|---|
| Motor | Day | k_{θ} (N · m) | k_{θ} (N · m) | Difference |
| N1 | Day 1 | 113.34 | 125.18 | 9.46% |
| N1 | Day 2 | 101.50 | 125.18 | 18.9% |
| N2 | Day 2 | 120.80 | 125.18 | 3.50% |
| N3 | Day 2 | 109.27 | 125.18 | 12.7% |

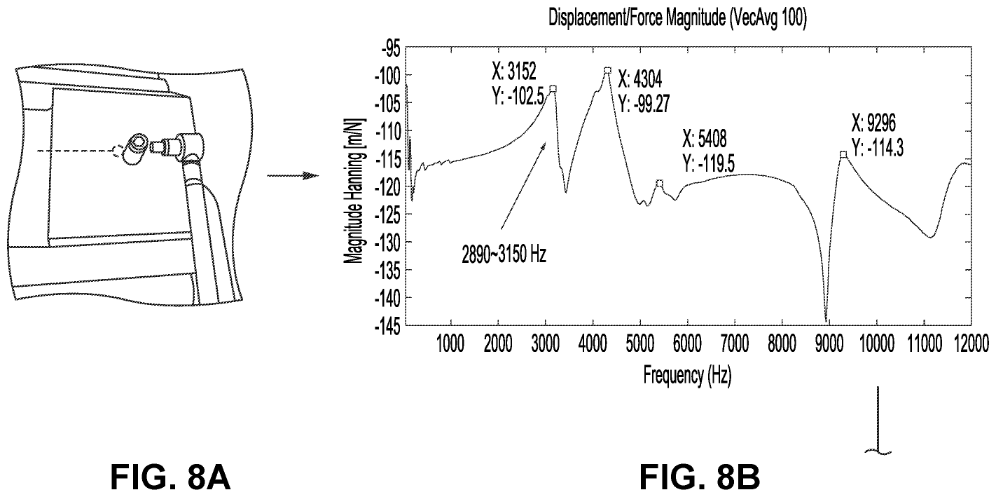

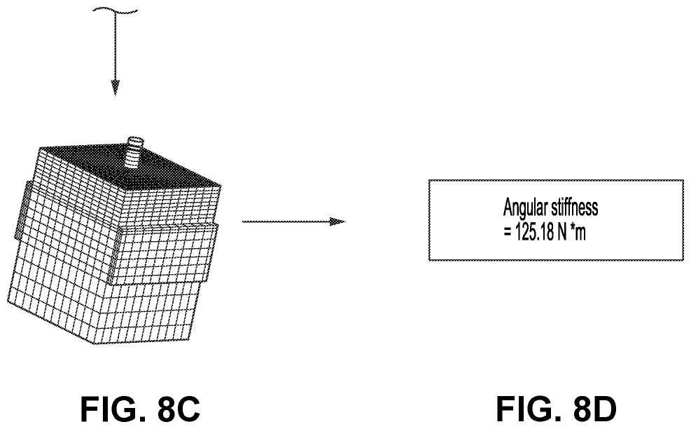

FIGS. 8A-8D illustrate the process flow of the benchmarking process. The first step is to use an impact hammer to excite the abutment of the implant system. At the tip of the impact hammer, there is a load cell to record the impact force f(t), as shown in FIG. 8A. As a result, the abutment vibrates and a laser Doppler vibrometer (LDV) measures the velocity v(t) of the abutment. The impact hammer and the LDV are located at 5.56 mm and 5.36 mm from the Sawbones®, respectively. Moreover, the implant test model was clamped in a vice at the same location.

The second step is to obtain a frequency response function of the implant test model. This was done by sending the force f(t) and velocity v(t) to a spectrum analyzer. FIG. 8B shows a sample frequency response function. Since the frequency response function was measured multiple times in multiple days, its first resonance frequency varied between 2890 Hz and 3150 Hz. This variation arose because the clamp force could not be well controlled and could vary from experiments to experiments.

The third step is model identification by matching the resonance frequency. A high-fidelity finite element model is created to simulate the implant test model, as shown in FIG. 8C. The laminated Sawbones® structure is modeled, with material properties (elastic moduli and density) obtained from manufacturer's data sheets. To model the variations in clamp force, two layers of buffer materials are introduced. The Young's modulus of the buffer materials was chosen to vary between 180 MPa and 320 MPa, so that the first resonance frequency would vary from 2890 Hz to 3150 Hz to match the experimental measurements. Therefore, the finite element model accurately reflects the implant test model in terms of the resonance frequency.

The fourth step is to predict the angular stiffness k_{θ}. Similar to FIG. 2C, a unit moment is applied to the model in FIG. 8C to secure the angle of rotation of the abutment, thus finding the angular stiffness k_{θ}. The angular stiffness extracted this way is 125.18 N·m, as shown in FIG. 8D. The benchmarked angular stiffness k_{θ} is also plotted in FIG. 7 for comparison. The angular stiffness k_{θ} measured from the stability tester agrees well with the benchmarked angular stiffness. The difference is roughly 4% for the best-case scenario and 20% for the worst-case scenario; see Table 1.

FIG. 9 is a block diagram of an example method for measuring a stability of an implant system. Method 300 shown in FIG. 9 presents an embodiment of a method that could be used by the device 200 as described above, as examples. Method 300 may include one or more operations, functions, or actions as illustrated by one or more of blocks 302-308. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 300 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 302, the method 300 includes positioning a device adjacent the implant system. The device may comprise the device 200 of any of the embodiments described above. In particular, the device 200 may include (i) a housing 202, (ii) an actuator 204 coupled to the housing 202, and (iii) a motion sensor 206 coupled to the housing 202. At block 304, the method 300 includes actuating the actuator 204 of the device 200 to vibrate the implant system. At block 306, the method 300 includes determining, via the motion sensor 206, motion data of the implant system. At block 308, the method 300 includes determining an angular stiffness of the implant system based on the motion data.

In one example, the step of determining the angular stiffness of the implant system comprises (i) receiving, via a controller of the device, the motion data of the implant system, (ii) transmitting, via the controller, the motion data to a user interface, and (iii) determining, via the user interface, the angular stiffness of the implant system based on the motion data.

In another example, the motion data comprises acceleration data, and the step of determining the angular stiffness of the implant system comprises (i) applying a nonlinear regression algorithm to extract a frequency ω and an amplitude A₀ of the acceleration data, (ii) determining an experimentally measured flexibility (C_{AB})ₑₓₚ of the implant system using the equation

$${( {C}_{AB} )}_{exp} = \frac{1}{{ω}^{2}} ( {A}_{0} / {F}_{0} ) ,$$

wherein F₀ is an amplitude of an actuator force, and (iii) determining the angular stiffness (k_{θ}) using the interpolation

$${k}_{θ} = {( {k}_{θ} )}_{i} + \frac{{( {C}_{AB} )}_{exp} - {( {C}_{AB} )}_{i}}{{( {C}_{AB} )}_{i + 1} - {( {C}_{AB} )}_{i}} [ {( {k}_{θ} )}_{i + 1} - {( {k}_{θ} )}_{i} ] ,$$

wherein (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A device for measuring a stability of an implant system, the device comprising:
a housing configured to be positioned adjacent the implant system;
an actuator coupled to the housing, wherein the actuator is configured to vibrate the implant system when actuated;
a motion sensor coupled to the housing; and
a controller in communication with the motion sensor and the actuator, wherein the controller includes at least one processor, and data storage including program instructions stored thereon that when executed by the at least one processor, cause the controller to perform functions including:
receiving motion data from the motion sensor when the actuator is actuated; and
determining an angular stiffness of the implant system based on the motion data.

2. (canceled)

3. The device of claim 1, wherein the implant system includes an implant implanted in a bone and an abutment coupled to the implant, wherein at least a portion of the abutment is exposed and not directly coupled to the bone, wherein the housing includes a cutout, and wherein the abutment is configured to be positioned at least partially within the cutout.

4. (canceled)

5. The device of claim 1, wherein the implant system includes a longitudinal axis extending from a first surface to a second surface opposite the first surface, wherein the implant system includes a second axis that is perpendicular to the longitudinal axis, and wherein the angular stiffness corresponds to a stiffness of a rotation of the implant system with respect to the second axis.

6. The device of claim 1, wherein the controller is further configured to:
provide a binary indication of whether or not the implant system is stable based on the determined angular stiffness of the implant system.

7. The device of claim 1, wherein the controller is further configured to:
provide a notification of a degree of stability of the implant system based on the determined angular stiffness of the implant system.

8. (canceled)

9. The device of claim 1, wherein the actuator is configured to vibrate at a first frequency which is measured by the motion sensor to define a first motion data, wherein the actuator is configured to vibrate at a second frequency which is measured by the motion sensor to define a second motion data, and wherein the controller determines the angular stiffness of the implant system based on both the first motion data and the second motion data.

10. The device of claim 1, wherein the actuator comprises a first actuator, the device further comprising:
a second actuator coupled to the housing, wherein the second actuator is configured to vibrate the implant system when actuated, wherein the first actuator is configured to vibrate at a first frequency which is measured by the motion sensor to define a first motion data, wherein the second actuator is configured to vibrate at a second frequency which is measured by the motion sensor to define a second motion data, and wherein the controller determines the angular stiffness of the implant system based on both the first motion data and the second motion data.

11. (canceled)

12. (canceled)

13. (canceled)

14. (canceled)

15. (canceled)

16. The device of claim 1, further comprising:
one or more bypass capacitors positioned between the motion sensor and the controller.

17. The device of claim 1, wherein the motion data comprises acceleration data, and wherein determining the angular stiffness of the implant system based on the acceleration data comprises:
applying a nonlinear regression algorithm to extract a frequency ω and an amplitude A₀ of the motion data;
determining an experimentally measured flexibility (C_{AB})ₑₓₚ of the implant system using the equation
$${( {C}_{AB} )}_{exp} = \frac{1}{{ω}^{2}} ( {A}_{0} / {F}_{0} ) ,$$
wherein F₀ is an amplitude of an actuator force; and
determining the angular stiffness (k_{θ}) using the interpolation
$${k}_{θ} = {( {k}_{θ} )}_{i} + \frac{{( {C}_{AB} )}_{exp} - {( {C}_{AB} )}_{i}}{{( {C}_{AB} )}_{i + 1} - {( {C}_{AB} )}_{i}} [ {( {k}_{θ} )}_{i + 1} - {( {k}_{θ} )}_{i} ] ,$$
wherein (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

18. A device for measuring a stability of an implant system, the device comprising:
a housing configured to be positioned adjacent the implant system;
an actuator coupled to the housing, wherein the actuator is configured to vibrate the implant system when actuated;
a motion sensor coupled to the housing; and
a controller in communication with the motion sensor and the actuator, wherein the controller includes at least one processor, and data storage including program instructions stored thereon that when executed by the at least one processor, cause the controller to perform functions including:
receiving motion data from the motion sensor when the actuator is actuated; and
transmitting the motion data to a user interface, wherein the user interface is configured to determine an angular stiffness of the implant system based on the motion data.

19. (canceled)

20. The device of any one of claims 18-19, wherein the implant system includes an implant implanted in a bone and an abutment coupled to the implant, wherein at least a portion of the abutment is exposed and not directly coupled to the bone, wherein the housing includes a cutout, and wherein the abutment is configured to be positioned at least partially within the cutout.

21. (canceled)

22. The device of claim 18, wherein the implant system includes a longitudinal axis extending from a first surface to a second surface opposite the first surface, wherein the implant system includes a second axis that is perpendicular to the longitudinal axis, and wherein the angular stiffness corresponds to a stiffness of a rotation of the implant system with respect to the second axis.

23. The device of claim 18, wherein the user interface is further configured to:
provide a binary indication of whether or not the implant system is stable based on the determined angular stiffness of the implant system.

24. The device of claim 18, wherein the user interface is further configured to:
provide a notification of a degree of stability of the implant system based on the determined angular stiffness of the implant system.

25. (canceled)

26. The device of claim 18, wherein the actuator is configured to vibrate at a first frequency which is measured by the motion sensor to define a first motion data, wherein the actuator is configured to vibrate at a second frequency which is measured by the motion sensor to define a second motion data, and wherein the user interface determines the angular stiffness of the implant system based on both the first motion data and the second motion data.

27. The device of claim 18, wherein the actuator comprises a first actuator, the device further comprising:
a second actuator coupled to the housing, wherein the second actuator is configured to vibrate the implant system when actuated, wherein the first actuator is configured to vibrate at a first frequency which is measured by the motion sensor to define a first motion data, wherein the second actuator is configured to vibrate at a second frequency which is measured by the motion sensor to define a second motion data, and wherein the user interface determines the angular stiffness of the implant system based on both the first motion data and the second motion data.

28. (canceled)

29. (canceled)

30. (canceled)

31. (canceled)

32. (canceled)

33. The device of claim 18, further comprising:
one or more bypass capacitors positioned between the motion sensor and the controller.

34. The device of claim 18, wherein the motion data comprises acceleration data, and wherein determining the angular stiffness of the implant system based on the acceleration data comprises:
applying a nonlinear regression algorithm to extract a frequency ω and an amplitude A₀ of the motion data;
determining an experimentally measured flexibility (C_{AB})ₑₓₚ of the implant system using the equation
$${( {C}_{AB} )}_{exp} = \frac{1}{{ω}^{2}} ( {A}_{0} / {F}_{0} ) ,$$
wherein F₀ is an amplitude of an actuator force; and
determining the angular stiffness (k_{θ}) using the interpolation
$${k}_{θ} = {( {k}_{θ} )}_{i} + \frac{{( {C}_{AB} )}_{exp} - {( {C}_{AB} )}_{i}}{{( {C}_{AB} )}_{i + 1} - {( {C}_{AB} )}_{i}} [ {( {k}_{θ} )}_{i + 1} - {( {k}_{θ} )}_{i} ] ,$$
wherein (k_{θ})ᵢ and (k_{θ})ᵢ₊₁ are angular stiffness predicted from a mathematical model of the implant system under two assumed elastic properties, whereas (C_{AB})ᵢ and (C_{AB})ᵢ₊₁ are respective flexibility predicted by the mathematical model with the two elastic properties.

35. A method for measuring a stability of an implant system, the method comprising:
positioning a device adjacent the implant system, wherein the device comprises (i) a housing, (ii) an actuator coupled to the housing, and (iii) a motion sensor coupled to the housing;
actuating the actuator of the device to vibrate the implant system;
determining, via the motion sensor, motion data of the implant system; and
determining an angular stiffness of the implant system based on the motion data, wherein the motion data comprises acceleration data, and wherein determining the angular stiffness of the implant system based on the acceleration data comprises:
applying an algorithm to extract a frequency ω and an amplitude A₀ of the motion data;
determining an experimentally measured flexibility (C_{AB})ₑₓₚ of the implant system using the equation
$${( {C}_{AB} )}_{exp} = \frac{1}{{ω}^{2}} ( {A}_{0} / {F}_{0} ) ,$$
wherein F₀ is an amplitude of an actuator force; and
determining the angular stiffness (k_{θ}) using an interpolation algorithm.

36. The method of claim 35, wherein determining the angular stiffness of the implant system comprises:
receiving, via a controller of the device, the motion data of the implant system;
transmitting, via the controller, the motion data to a user interface; and
determining, via the user interface, the angular stiffness of the implant system based on the motion data.

37. (canceled)
